(19) United States
(12) Patent Application Publication
(10) Pub. No.: US 2016/0101114 A1
(43) Pub. Date: Apr. 14, 2016

(54) ARTICLES AND METHODS FOR PREVENTING AND TREATING DERMATOLOGIC ADVERSE EVENTS

(71) Applicant: SLOAN-KETTERING INSTITUTE FOR CANCER RESEARCH, New York, NY (US)

(72) Inventors: Mario E. Lacouture, New York, NY (US); Ghassan Khaled Abou-Alfa, New York, NY (US)

(21) Appl. No.: 14/888,392

(22) PCT Filed: Mar. 24, 2014
(86) PCT No.: PCT/US14/31545

§ 371 Date: Oct. 30, 2015

Related U.S. Application Data
(60) Provisional application No. 61/804,907, filed on Mar. 25, 2013.

(51) Int. Cl. A61K 31/554; A61K 31/44; A61K 45/06; A61K 9/00; A61K 31/573

(52) U.S. Cl. CPC A61K 31/554; A61K 9/0014; A61K 31/573; A61K 45/06; A61K 31/44

(57) ABSTRACT

The present invention provides articles and methods for preventing or treating dermatologic adverse events.

### CROSS REFERENCE TO RELATED APPLICATIONS

The present invention claims priority to U.S. provisional patent application No. 61/804,907, filed Mar. 25, 2013, the entire contents of which are hereby incorporated by reference.

### SUMMARY OF THE INVENTION

The present invention provides, among other things, articles for preventing and treating dermatologic adverse events. In some embodiments, the present invention provides articles for preventing and treating hand foot syndrome. In some embodiments, a provided article comprises one or more calcium channel blockers and vasodialtors. In some embodiments, a calcium channel blocker in a provided article is continuously released. In some embodiments, the continuous release is achieved through matrix diffusion controlled system. In some embodiments, the present invention provides an article that is juxtaposed to a person's body, said article comprising:
1) an effective amount of a calcium channel blocker, vasodilator, or a pharmaceutically acceptable salt thereof; and
2) a matrix diffusion controlled system;
   wherein the matrix diffusion controlled system provides a continuous release of said calcium channel blocker, vasodilator, or a pharmaceutically acceptable salt thereof

In some embodiments, the present invention provides methods of preventing and treating a dermatologic adverse event caused by medical therapy, comprising contacting the skin at or adjacent to the site in need thereof with an article, said article comprising:
1) an effective amount of a calcium channel blocker, vasodilator, or a pharmaceutically acceptable salt thereof; and
2) a matrix diffusion controlled system;
   wherein the matrix diffusion controlled system provides a continuous release of said calcium channel blocker, vasodilator, or a pharmaceutically acceptable salt thereof

### BRIEF DESCRIPTION OF THE DRAWINGS

FIG. 1: Illustrative symptoms of hand foot syndrome.

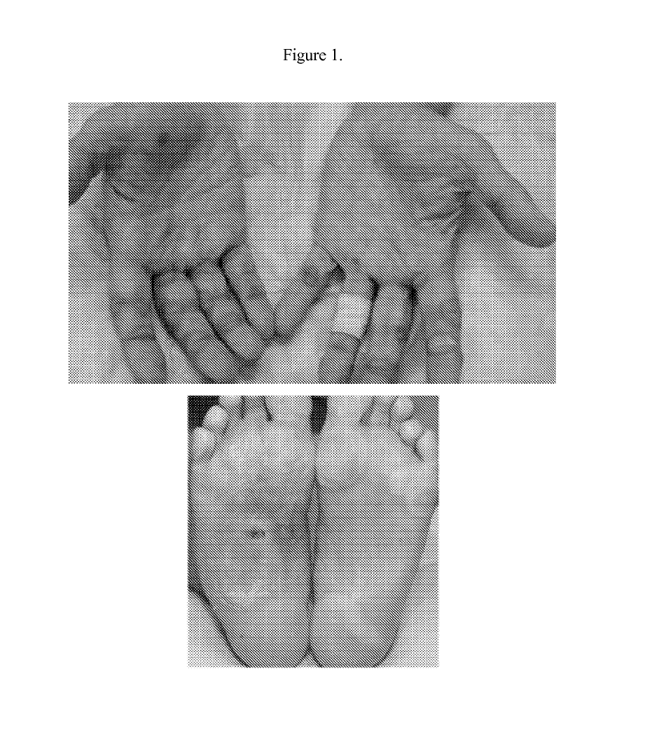

### DETAILED DESCRIPTION OF CERTAIN EMBODIMENTS

Therapies used against cancer and other conditions can cause multiple side effects and damages to normal cells. In addition to hypertension and diarrhea, skin toxicity is common. For cancer therapies comprising the use of multikinase inhibitors, one clinically significant dermatologic adverse event is hand foot syndrome, with an all grade incidence of 60% (sorafenib), 30% (sunitinib), pazopanib (5%), axitinib (29%), vandetanib (5%), ziv-aflibercept (2%), cabozantinib (50%), and regorafenib (46%). Hand foot syndrome induced by multikinase inhibitors is characterized by affecting the palms and soles and any other areas exposed to friction or trauma (elbows, knees). In some embodiments, it appears within the first 6 weeks in most patients, initially with painful blisters, followed after several months by thick, hyperkeratotic areas resembling calluses. Extremely painful lesions on the palms and soles usually occur in the fingertips, over the interphalangeal joints, and on the heels and forefeet, which may affect patients' ability to conduct their daily activities, and negatively affect their quality of life. Furthermore, this painful condition may result in inconsistent administration of anticancer therapies, which may affect clinical outcome. Skin biopsies demonstrate a band-like area of necrosis with an underlying inflammatory infiltrate, with ecstatic vessels and cystic degeneration of eccrine glands. Without the intention to be limited by theory, the pathogenic mechanisms are believed to occur as a result of peripheral vasoconstriction, which in addition to causing hypertension, result in decreased capillary density in the skin, especially in distal areas devoid of collateral flow, such as the hands and feet. In the setting of this decreased capillary flow due to medication-induced vasoconstriction, the skin in palms and soles is not able to receive the normal nourishment necessary for homeostasis and functioning, which results in necrosis and the painful blisters that are observed.

Conventional treatment for multikinase inhibitor-induced hand foot syndrome includes topical (for grades ½) or a combination of topical and oral agents (for grades 3) against the main symptom of pain. Prior to beginning therapy, removal of any calluses and minimizing any foot malalignment with the help of a podiatrist or orthotist is key, as well as the use of thick socks, gloves, and soft slippers or shoes. The prophylactic use of daily moisturizers containing urea 10% three times a day has been shown to decrease all grade hand foot syndrome to sorafenib by about 20%. For painful blisters, using high potency topical corticosteroids (clobetasol, betamethasone) and topical anesthetics (lidocaine, prilocaine/lidocaine, lidocaine patches) are helpful. For hyperkeratotic (callused) areas, keratolyitic moisturizers (salicylic acid 6%, urea 40%, ammonium lactate 12%) are helpful to soften and thin these lesions when used several times a day. Since pain is the most important symptom, oral analgesics (opioids, NSAIDs) should be considered.

However, the use of topical compositions for treatment of skin toxicities, for example, hand foot syndrome, has its problems. Despite the partial benefit of these interventions, none of them function at the level of the inciting mechanisms leading to the toxicity, namely, vasoconstriction in the palms and soles. Moreover, the use of topical compositions such as cream in the palms and soles is problematic, since hand washing, sweating, and the use of cotton gloves or socks will decrease the amount of agent that is in contact with the skin. In some embodiments, the present invention provides articles and methods for solving these problems.

The present invention encompasses the recognition that improved administration of topical compositions offers better outcomes for the treatment of certain skin toxicities or disorders. In some embodiments, the present invention provides an article that is juxtaposed to a person's body, wherein said article comprises an effective amount of a drug for prevention or treatment of skin toxicity. In some embodiments, the present invention provides an article that is juxtaposed to a person's body, wherein said article comprises an effective amount of a drug for prevention or treatment of hand foot syndrome. In some embodiments, the article delivers the drug in a continuous fashion. In some embodiments, the present invention provides an article that is juxtaposed to a person's body, wherein said article comprises an effective amount of a calcium channel blocker drug or its pharmaceutically acceptable salt thereof. In some embodiments, the present invention provides an article that is juxtaposed to a person's body, wherein said article comprises an effective amount of a calcium channel blocker drug or its pharmaceutically acceptable salt thereof, and delivers the calcium channel blocker drug continuously. In some embodiments, the present invention provides an article that is juxtaposed to a person's body, said article comprising:
1) an effective amount of a calcium channel blocker drug or its pharmaceutically acceptable salt thereof; and
2) a matrix diffusion controlled system;
   wherein the matrix diffusion controlled system provides a continuous release of said calcium channel blocker drug or its pharmaceutically acceptable salt thereof.

In some embodiments, the present invention provides methods for preventing or treating skin toxicity. In some embodiments, the present invention provides methods for preventing or treating skin toxicity caused by cancer therapies. In some embodiments, the present invention provides methods for preventing or treating a dermatologic adverse event caused by cancer therapy. In some embodiments, the skin toxicity or dermatologic adverse event is hand foot syndrome.

In some embodiments, the present invention provides methods for preventing or treating a dermatologic adverse event caused by medical therapy, comprising contacting the skin at or adjacent to the site in need thereof with an article, said article comprising:
1) an effective amount of a calcium channel blocker drug or its pharmaceutically acceptable salt thereof; and
2) a matrix diffusion controlled system;
   wherein the matrix diffusion controlled system provides a continuous release of said calcium channel blocker drug or its pharmaceutically acceptable salt thereof. In some embodiments, the dermatologic adverse event is hand foot syndrome.

In some embodiments, the method is characterized in that the drug contacts the skin after the article is placed on the patient's body. In some embodiments, medical therapy is cancer therapy.

In some embodiments, an article is a garment, footwear, or hand wear. In some embodiments, an article is a garment. In some embodiments, an article is footwear. In some embodiments, the article is hand wear. In some embodiments, footwear is a sock, stocking, shoe, sneaker, shoe insole or shoe lining. In some embodiments, hand wear is a glove. It is understood the article, such as garment, footwear or hand wear, can be made of different type of materials, including but limited to both natural and non-natural polymers, and the combinations thereof. In some embodiments, an article is made of textiles. In some embodiments, an article is made of cotton, optionally having an inner liner comprising a non-cotton material. In certain embodiments, an article made of a material substantially other than cotton.

Various calcium channel blocker drug or its pharmaceutically acceptable salt thereof can be used in the article. In some embodiments, a calcium channel blocker drug is, diltiazem, lidocaine, isosorbide dinitrate, or any combination thereof. In some embodiments, a calcium channel blocker drug is nitroglycerin. In some embodiments, a calcium channel blocker drug is diltiazem. In some embodiments, a calcium channel blocker drug is diltiazem HCl. In some embodiments, a calcium channel blocker drug is isosorbide dinitrate. In some embodiments, an article comprises a combination of calcium channel blocker drugs or their pharmaceutically acceptable salts thereof. In some embodiments, an article comprises a combination of calcium channel blocker drugs or their pharmaceutically acceptable salts thereof, wherein the calcium channel blocker drug is diltiazem, lidocaine, or isosorbide dinitrate. In some embodiments, a calcium channel blocker drug is lidocaine.

In some embodiments, a vasodialator drug is minoxidil, tadalafil, sildenafil, or nitroglycerin.

In some embodiments, a provided article can be exposed to, coated with, or impregnated with a composition comprising a calcium channel blocker. In some embodiments, a provided article can be treated with a calcium channel blocker, for example, by spraying, imprinting, washing, injecting, coating, or other method of application. In some embodiments, articles are suitable for one use only. In other embodiments, articles are suitable for multiple use. In some embodiments, an article suitable for multiple use allows for a calcium channel blocker to be reapplied, refilled, etc., such that the release of the drug may continue.

Various materials can be used as a matrix diffusion controlled system in a provided article. Matrix diffusion systems are known in the art and readily ascertainable and prepared by the skilled artisan. For example, in some embodiments a matrix diffusion controlled system contains a drug reservoir comprising a homogenous dispersion of drug particles in a polymer matrix. In some embodiments, a matrix diffusion controlled system comprises polymeric materials. In some embodiments, a matrix diffusion controlled system comprises natural polymers. In some embodiments, a matrix diffusion controlled system comprises non-natural polymers. In some embodiments, a matrix diffusion controlled system comprises a combination of natural and non-natural polymers. In some embodiments, a polymer is hydrophilic. In some embodiments, a polymer is hydrophobic. In some embodiments, a matrix diffusion controlled system comprises a mixture of hydrophobic and hydrophilic polymers. Exemplary polymers include but are not limited to hydroxypropyl methylcellulose (HPMC), polyvinyl pyrrolidone (PVP), Eudragit E100, Eudragit L100, polyethylene glycol, ethylcellulose (EC) and their combinations thereof. In some embodiments, a matrix diffusion controlled system comprises HPMC and EC. In some embodiments, a matrix diffusion controlled system comprises PVP and EC.

In some embodiments, an article further comprises one or more additives such as plasticizers or dispersants. In some embodiments, one or more of plasticizers are phthalate esters. In some embodiments, a plasticizer is dibutyl phthalate. In some embodiments, a plasticizer is triethyl citrate.

In some embodiments, an article further comprises one or more penetration enhancers. In some embodiments, an enhancer is isopropyl myristate (IPM), isopropyl palmitate (IPP), N-methyl-2-pyrrolidone, oleic acid, polyethylene glycol 400, propylene glycol, and/or Tween 80.

In some embodiments, an article provides a continuous release of a calcium channel blocker drug for more than about 1, 2, 4, 6, 8, 10, 12, 18, 24, 48 or 72 hours. In some embodiments, an article provides a continuous release of a calcium channel blocker drug for more than about 1, 2, 3, 4, 5, 6, 7, 14 or more days.

In some embodiments, a skin toxicity or dermatologic adverse event that a person suffers from or is susceptible to is caused by cancer therapy. In some embodiments, the skin toxicity or dermatologic adverse event is hand foot syndrome. In some embodiments, the cancer therapy comprises blocking a cancer-related protein. In some embodiments, the cancer therapy comprises the blocking of VEGFR. In some embodiments, the cancer therapy comprises the blocking of PDGFR. In some embodiments, the cancer therapy comprises the use of one or more kinase inhibitors. In some embodiments, the kinase inhibitor is a multikinase inhibitor. In some embodiments, the inhibitor is sorafenib, sunitinib, pazopanib, cabozantinib, ziv-aflibercept, vandetanib, axitinib, or regorafenib, or its pharmaceutically acceptable salt thereof. In some embodiments, the inhibitor is sorafenib. In some embodiments, the inhibitor is sunitinib. In some embodiments, the inhibitor is pazopanib. In some embodiments, the inhibitor is axitinib. In some embodiments, the inhibitor is regorafenib. In some embodiments, the inhibitor is vandetanib. In some embodiments, the inhibitor is cabozantinib. In some embodiments, the inhibitor is ziv-aflibercept. In some embodiments, a combination of different kinase inhibitors is used. In some embodiments, the cancer therapy optionally comprises radiotherapy. In some embodiments, the cancer therapy optionally comprises one or more of thyroidectomy, radioactive iodine, cyberknife, everolimus, sodium iodide I-131, AZD6244, iodine I-124, capecitabine, oxaliplatin, gemcitabine, doxorubicin, carboplatin, VEGF trap, megestrol, and/or vandetanib.

In some embodiments, the cancer is advanced renal cell carcinoma. In some embodiments, the cancer is gastrointestinal stromal tumor. In some embodiments, the cancer is pancreatic neuroendocrine tumor. In some embodiments, the cancer is liver cancer. In some embodiments, the cancer is medullary thyroid cancer.

In some embodiments, the article further comprises one or more moisturizers. In some embodiments, the article further comprises urea.

In some embodiments, an article comprises a calcium channel blocker or vasodialator as part of a pharmaceutical composition. In some embodiments, an article comprises a calcium channel blocker or vasodialator in combination with a moisturizer. In some embodiments, a moisturizer is a suitable hand or body lotion.

In some embodiments, the article further comprises one or more vasodilators. In some embodiments, a vasodilator is selected from nitroglycerin, tadalafil, sildenafil, or minoxidil. In some embodiments, a vasodilator is tadalafil or sildenafil.

In some embodiments, the article further comprises one or more corticosteroids. In some embodiments, the article further comprises clobetasol. In some embodiments, the article further comprises betamethasone.

In some embodiments, the article further comprises one or more keratolyitic moisturizers. Exemplary keratolyitic moisturizers includes but are not limited to salicylic acid, urea, ammonium lactate, or combinations thereof.

In some embodiments, moisturizers, corticosteroids and oral analgesics, such as opioids and NSAIDs, are optionally administered prior to, concurrently with, or subsequent to the provided article. In some embodiments, other treatments, such as removal of calluses and minimizing foot malalignment, are administered prior to, concurrently with, or subsequent to the provided article.

Hand foot syndrome is characterized by different grades as exemplified in Table 1, below (Blum et al, J. Clin. Oncol. 1999, 17(2), 485-93):

TABLE 1
Hand Foot Syndrome Grading Scale

| Grade | Clinical Domain* | Functional Domain |
|---|---|---|
| 1 | Minimal skin changes or | Does not limit ADL |
| | dermatitis (e.g., erythema, | |
| | edema, or hyperkeratosis) without pain | |
| 2 | Skin changes (e.g., peeling, | Limiting instrumental |
| | blisters, bleeding, edema, or | ADL |
| | hyperkeratosis) with pain; | |
| 3 | Severe skin changes (e.g., | Limiting self care |
| | peeling, blisters, bleeding, | ADL |
| | edema, or hyperkeratosis) with | |
| | pain; | |

*In case of discrepancy between the clinical and functional domains, the assigned grade should correspond to the most important intensity from one or the other domain. Activities of Daily Living (ADL): Instrumental ADL refers to preparing meals, shopping for groceries or clothes, using the telephone, managing money, etc. Self care ADL refers to bathing, dressing and undressing, feeding self, using the toilet, taking medications, and not bedridden.

After being administered to a subject suffering from hand foot syndrome, in some embodiments, a provided article or method maintains or decreases the grade of the hand foot syndrome of said subject. In some embodiments, the grade is maintained at or decreased to grade 1 or lower. In some embodiments, the grade is maintained at or decreased to grade 1. In some embodiments, the grade is maintained at or decreased to grade 0 (i.e., substantially no hand foot syndrome remains). In some embodiments, the grade is maintained at or decreased to grade 2 or lower. In some embodiments, the grade is maintained at or decreased to grade 2. In some embodiments, the grade is decreased from grade 3 to grade 2, 1, or 0. In some embodiments, the grade is decreased from grade 3 to grade 2 or 1. In some embodiments, the grade is decreased from grade 3 to grade 1 or 0. In some embodiments, the grade is decreased from grade 3 to grade 2. In some embodiments, the grade is decreased from grade 3 to grade 1. In some embodiments, the grade is decreased from grade 3 to grade 0. In some embodiments, the grade is decreased from grade 2 to grade 0 or grade 1. In some embodiments, the grade is decreased from grade 2 to grade 1. In some embodiments, the grade is decreased from grade 2 to grade 0. In some embodiments, the grade is decreased from grade 1 to grade 0.

In some embodiments, a provided article or method prevents the progression of hand foot syndrome. In some other embodiments, a provided article or method stops the progression of hand foot syndrome. In some embodiments, a provided article or method slows the progression of hand foot syndrome.

In some embodiments, a provided article or method is used prophylactically. In some embodiments, a provided article or method is used before the onset of hand foot syndrome. In some embodiments, a provided article or method prevents the onset of hand foot syndrome. In some embodiments, a provided article or method delays the onset of hand foot syndrome.

A provided article or method brings multiple benefits to cancer therapy. Among other things, in some embodiments, a provided article or method prevents or minimizes undesirable modifications of a cancer therapy, including but not limited to the dosage decrease of one or more drugs or treatments, the switch from one drug or treatment to another, or the decrease of time a subject can be treated. In some embodiments, a provided article or method enables a patient to continue one or more cancer therapies for a period of time greater than the patient could without a provided article or method. In some embodiments, a provided article or method increases the benefits of one or more drugs or treatments. In certain embodiments, a provided article or method allows for uninterrupted cancer treatment such that the survival rate of a patient or patient population increases compared to the survival rate without a provided article or method. In certain embodiments, a provided article or method allows for uninterrupted cancer treatment such that a patient's or patient population's life term is extended compared to the term without a provided article or method. In certain embodiments, a provided article or method allows for uninterrupted cancer treatment such that the cancer being treated enters remission at a rate or frequency greater than in the absence of the article or method.

A provided article or method improves the life quality of a subject suffering from or susceptible to hand foot syndrome. In some embodiments, a provided article or method provides better medical outcomes. In some embodiments, a provided article or method provides better mobility. In some embodiments, a provided article or method relieves pain caused by hand foot syndrome. In some embodiments, a provided article or method improves the quality of life whilst on cancer treatments.

### DEFINITIONS

In some embodiments, the present disclosure provides “pharmaceutically acceptable” compositions, which comprise a therapeutically effective amount of one or more of the compounds described herein, formulated together with one or more pharmaceutically acceptable carriers (additives) and/or diluents. As described in detail, the pharmaceutical compositions of the present disclosure may be specially formulated for administration in solid or liquid form, including those adapted for the following: topical application, for example, as a cream, ointment, or a controlled-release patch or spray applied to the skin, lungs, or oral cavity; intravaginally or intrarectally, for example, as a pessary, cream or foam; ocularly; transdermally; or nasally, pulmonary and to other mucosal surfaces.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein and in the claims, the singular forms “a”, “an”, and “the” include the plural reference unless the context clearly indicates otherwise. Thus, for example, a reference to “a compound” includes a plurality of such compounds.

The term “palliative” refers to treatment that is focused on the relief of symptoms of a disease and/or side effects of a therapeutic regimen, but is not curative.

As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition for the purpose of decreasing the risk of developing pathology associated with the disease, disorder, and/or condition.

The expression “unit dose” as used herein refers to a physically discrete unit of a formulation appropriate for a subject to be treated. It will be understood, however, that the total daily usage of a formulation of the present invention will be decided by the attending physician within the scope of sound medical judgment. The specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts. A particular unit dose may or may not contain a therapeutically effective amount of a therapeutic agent.

An individual who is “suffering from” a disease, disorder, and/or condition has been diagnosed with and/or displays one or more symptoms of the disease, disorder, and/or condition.

An individual who is “susceptible to” a disease, disorder, and/or condition has not been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not necessarily develop the disease, disorder, and/or condition. In some embodiments, an individual who is susceptible to a disease, disorder, and/or condition is genetically prone to the disease, disorder, and/or condition.

### Exemplification

Calcium channel blocker drugs, optionally with other components described above and herein, have been test on patient for their use in the articles and methods in the present invention and are described below in Table 2.

For example, diltiazem is a nondihydropyridine (non-DHP) member of the class of drugs known as calcium channel blockers, used in the treatment of hypertension, angina pectoris, and some types of arrhythmia. Topical diltiazem causes vasodilation when applied topically, and is superior to other non-surgical interventions for anal fissures. In addition, diltiazem can be delivered transdermally with the use of a matrix diffusion controlled system, to achieve continuous release. Based on these observations, Applicant used diltiazem cream to treat patients suffering from hand foot syndrome. Applicant observed that the use of this cream reduces the pain and the need to decrease and/or discontinue the anticancer medicine. The use of diltiazem or other calcium channel blockers in the articles and methods described herein provides even more beneficial therapeutic effects than the use of topical cream(s) alone.

TABLE 2
Exemplary calcium channel blocker treatment for hand foot syndrome

| | | | | Anticancer | HFSR | | HFSR |
|---|---|---|---|---|---|---|---|
| # | Age | Gender | Cancer Diagnosis | Therapy | Grade | Intervention | Grade |
| 1 | 57 | M | Metastatic thyroid | Thyroidectomy, | 2 | Diltiazem 2%, | 1 |
| | | | cancer | Radioactive iodine, | | Carmol 40 cream, | |
| | | | | Cyberknife, | | Clobetasol cream, | |
| | | | | Sunitinib, | | Ped Egg for | |
| | | | | Sorafenib, | | calluses, | |
| | | | | Everolimus | | Soft shoes, | |
| | | | | | | Cotton gloves | |
| 2 | 60 | M | Thyroid cancer | Sodium Iodide I- | 3 | Diltiazem cream, | 1 |
| | | | | 131, | | Clobetasol Spray, | |
| | | | | AZD6244, | | Urea 40% | |
| | | | | Iodine I-124, | | | |
| | | | | Sorafenib, | | | |
| | | | | Everolimus, | | | |
| | | | | Thyroidectomy | | | |
| 3 | 61 | M | Thyroid cancer | Everolimus, | NA | Diltiazem cream, | 0 |
| | | | | Sorafenib, | | Urea cream | |
| | | | | Thyroidectomy | | (prophylactically) | |
| 4 | 55 | F | Thyroid cancer | Pazopanib, | 2 | Urea cream 40%, | 1 |
| | | | | Everolimus, | | Diltiazem 2% | |
| | | | | Sorafenib, | | | |
| | | | | Sodium Iodide I-131 | | | |
| 5 | 42 | M | Metastatic esophageal | Xeloda/oxaliplatin, | 3 | Urea cream 40%, | 1 |
| | | | cancer | Xeloda/Gemcitabine, | | Diltiazem 2% | (within 3-5 days) |
| | | | | Sorafenib | | | |
| 6 | 69 | M | Metastatic medullary | Everolimus, | 1 | Urea cream 40%, | 1 |
| | | | thyroid cancer | Sorafenib | | Diltiazem 2%, | (later flared to 3, |
| | | | | | | Clobetasol needed | sorafenib dose |
| | | | | | | to be added to the | reduced - got |
| | | | | | | above 2 creams to | controlled, later |
| | | | | | | control blistering | waxing/waning at |
| | | | | | | | 1) |
| 7 | 58 | M | RCC | Sunitinib | 2 | Urea cream 40%, | 1 |
| | | | | | | Diltiazem 2%, | |
| | | | | | | Clobetasol needed | |
| | | | | | | to be added to the | |
| | | | | | | above 2 creams to | |
| | | | | | | control blistering | |
| 8 | 51 | F | Thyroid cancer | Doxorubicin, | 3 | Urea cream 40%, | 1 |
| | | | | Carboplatin, | | Diltiazem 2% | |
| | | | | Pazopanib, | | (Sorafenib also | |
| | | | | Everolimus, | | held), | |
| | | | | VEGF Trap | | later Clobetasol | |
| | | | | | | added, | |
| | | | | | | Ped Egg | |
| 9 | 64 | F | Thyroid cancer | Megestrol, | NA | Diltiazem cream | 0 |
| | | | | Everolimus, | | (prophylactically) | |
| | | | | Sorafenib, | | | |
| | | | | later Vandetanib | | | |

1. An article that is juxtaposed to a patient's body, said article comprising:
1) an effective amount of a calcium channel blocker, vasodilator, or a pharmaceutically acceptable salt thereof; and
2) a matrix diffusion controlled system;
wherein the matrix diffusion controlled system provides a continuous release of said calcium channel blocker, vasodilator, or a pharmaceutically acceptable salt thereof.

2. The article of claim 1, wherein the article is a garment, footwear, or hand wear.

3. The article of claim 2, wherein the footwear is a sock, stocking, shoe, sneaker, shoe insole, or shoe lining.

4. The article of claim 2, wherein the hand wear is a glove.

5. The article of claim 1, wherein the calcium channel blocker drug is diltiazem, lidocaine, or isosorbide dinitrate.

6. The article of claim 1, wherein the calcium channel blocker is diltiazem.

7. The article of claim 1, wherein the matrix diffusion controlled system comprises polymeric materials.

8. The article of claim 7, wherein the polymeric material is hydroxypropyl methylcellulose (HPMC), polyvinyl pyrrolidone (PVP), Eudragit E100, Eudragit L100, polyethylene glycol, ethylcellulose (EC), or combinations thereof.

9. The article of claim 7, wherein the calcium channel blocker is dispersed in the matrix diffusion controlled system.

10. The article of claim 1, further comprising an additive.

11. The article of claim 1, further comprising a penetration enhancer.

12. The article of claim 1, further comprising one or more moisturizers and/or corticosteroids.

13. The article of claim 1, wherein the continuous release of the calcium channel blocker drug last for more than about 1, 2, 4, 6, 8, 10, 12, 18 hours, or more than about 1, 2, 3, 4, 5, 6, 7, or 14 days

14. A method of preventing or treating a dermatologic adverse event caused by medical therapy, comprising contacting the skin at or adjacent to the site in need thereof with the article of claim 1.

15. A method of preventing or treating a dermatologic adverse event caused by medical therapy, comprising contacting the skin with a vasodilator.

16. The method of claim 14, wherein the dermatologic adverse event is hand foot syndrome.

17. The method of claim 14, wherein the medical therapy comprises the use of a kinase inhibitor.

18. The method of claim 17, wherein the kinase inhibitor is a multikinase inhibitor.

19. The method of claim 17, wherein the kinase inhibitor is sorafenib, sunitinib, pazopanib, axitinib, cabozantinib, ziv-aflibercept, or regorafenib.

20. The method of claim 1, wherein the site is a body area exposed to friction or trauma.

21-34. (canceled)
